(19) United States
(12) Patent Application Publication
(10) Pub. No.: US 2014/0259904 A1
(43) Pub. Date: Sep. 18, 2014

(54) SYSTEM AND PROCESS FOR IRRIGATING AND MONITORING THE GROWTH OF PLANTS

(71) Applicant: William Charles Collard, Plandome, NY (US)

(72) Inventor: William Charles Collard, Plandome, NY (US)

(21) Appl. No.: 13/844,354

(22) Filed: Mar. 15, 2013

(51) Int. Cl. A01G 27/00

(52) U.S. Cl. CPC A01G 27/00
USPC 47/48.5

(57) ABSTRACT

There is at least one embodiment that comprises a system for harvesting plants comprising at least one container for receiving and growing plants; at least one irrigation system configured to feed water to at least one of said plurality of containers; and at least one microprocessor configured to calculate a time until reaching a harvest point based upon a position of the plants. The at least one container can comprise a plurality of containers. The at least one irrigation system comprises at least one level valve configured to close when fluid in at least one container of said plurality of containers reaches a predetermined level.

### BACKGROUND

At least one embodiment of the invention relates to a system and process for monitoring the growth of plants. Traditionally one would plant a garden in a person's backyard. That person would then have to rely on natural irrigation such as rain, or rising water tables. Alternatively the user would have to rely on going out and watering the garden as well to make sure that the plants have received enough water. However, there is no known system which tracks and measures as well as delivers irrigation as well as indicates the types of plants that should be planted in a particular region as well as tracks the progress of growth of these plants.

### SUMMARY

There is at least one embodiment that comprises a system for harvesting plants comprising at least one container for receiving and growing plants; at least one irrigation system configured to feed water to at least one of said plurality of containers; and at least one microprocessor configured to calculate a time until reaching a harvest point based upon a position of the plants. The at least one container can comprise a plurality of containers. The at least one irrigation system comprises at least one level valve configured to close when fluid in at least one container of said plurality of containers reaches a predetermined level. The at least one microprocessor can be configured to perform the following steps: receive and process information about the geographic location of said at least one container; receive and process information about the time of year of year; determine a type of at least one plant that should be planted in said at least one container based upon said information relating to geographic location and information relating to the time of the year for planting.

The processor can be configured to determine the length of time that said at least one plant will last until a preset harvest point. In at least one embodiment, the preset harvest point is a point at which the plant is to be replanted in another container. The least one irrigation system can further comprise at least one solar panel configured to control an electronics switching system for switching on or off a valve for delivering fluid to at least one container. The at least one irrigation system further comprises at least one pump for pumping water through the system.

### BRIEF DESCRIPTION OF THE DRAWINGS

Other objects and features of the present invention will become apparent from the following detailed description considered in connection with the accompanying drawings which discloses at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a plan view of a device for offering a watering system for use in growing plants;

FIG. 2 is a second plan view of the device for providing a watering system for use in growing plants and for monitoring the growth in plants

FIG. 3A is a side view of a first embodiment of an irrigation system;

FIG. 3B is an end view of the embodiment shown in any one of FIG. 3A or FIG. 4;

FIG. 4 is a side view of another embodiment of an irrigation system;

FIG. 5 is a side view of a container for a watering system top view of an indoor irrigation system;

FIG. 6A shows a side view of a base connector 50 which is configured to clamp onto an extension or line 201a to secure a container or a container holder thereto;

FIG. 6B there is an additional connector which is configured to connect to a container;

FIG. 6C shows a side cross-sectional view of the device;

FIG. 6D shows a side view of another type of securing configuration wherein a connector element 56 is positioned offset from another connector element 52;

FIG. 7A shows a top view of another embodiment of container;

FIG. 7B is a top view of another embodiment;

FIG. 8 is a side view of the connection system of the container as well as the fluid flow system;

FIG. 9A shows a side view of valve which includes turning valve and extension;

FIG. 9B shows a side view of a three way valve;

FIG. 9C shows a quick connect version which shows quick connects for connecting to an extension;

FIG. 10A is a side view of another embodiment;

FIG. 10B is a side view of another embodiment;

FIG. 10C is a front view of the support which shows a conduit, supported by a bracket;

FIG. 11 is a side view of an indoor tray which can be used to grow plants in an initial type system;

FIG. 12A shows a side view of an opening or hole which is configured to receive a cartridge or similar type of element which is configured to hold a plant seedling or herb sprout;

FIG. 13 shows a side view of the portable tray system shown in FIG. 11;

FIG. 14 shows a schematic diagram of a computer system for controlling or using the system;

FIG. 15A shows a schematic block diagram for the electrical components of a computer system;

FIG. 15B shows another schematic block diagram for the electrical components of a computer system;

FIG. 16 is a flow chart for the process for selecting the plants or types of plants to plant; and

FIG. 17 is a flow chart for tracking the growth of plants in the system.

### DETAILED DESCRIPTION

FIG. 1 is a plan view of an aspect of the invention 10 which shows a plot of land such as plot 5 having a house 11 disposed thereon. This view shows extensions 20a, 20b, and 20c which are configured to provide support for irrigation or hydroponic containers such as containers 20a, or 220. There is at each end of each extension a controller 30a, 30b, and 30c. Each extension can be in the form of a fence line of piping or a simple extension of irrigation piping which extends towards or through a series of containers which are shown in FIG. 2. The design of these extensions 20a, 20b, and 20c can be in any suitable shape or form and can be in any order. The positioning of these extensions can be in any form as well. For example, extension 20a can be positioned as diagonal across a yard, or positioned across the front of a house 11 as well. These extensions can be in the form of hose or steel piping which extends across a yard or a field and which is of suitable gauge to support containers 22a etc. In at least one embodiment, the extension such as any one of extension 20a, 20b, and 20c can comprise a fence suitable to carry a plurality of containers such as containers 22a. This fence can be made of any suitable material such as wood, polyvinyl chloride (PVC) any suitable metal such as steel, aluminum, or copper or any other suitable material. The fence can be formed separate from a water conveying system or can be formed integral with a water conveying system. In at least one embodiment these extensions can be configured to be a conduit for water or fluid or simply be configured to hold a container such as a container 22a or 220.

FIG. 2 is a plan view of another version which shows house 11, with extensions 20a, 20b, 20c extending out on an outer periphery. A plurality of photovoltaic cells or solar panels 31 are positioned around the perimeter of the house as well. These photovoltaic cells are configured to be a preset size so that the energy produced by these cells are configured to provide a central computer such as computer 420 with information relating to the amount of sunlight produced on a particular plot of land such as plot 5. Three is also another set of extensions 20d, 20e, and 20f showing that these extensions can be positioned at different positions in a plot and even nested inside of one another. In this view there is an irrigation system 39 which can include a fluid pumping controller 39a, a rain sensor 39b and a fluid pump 39c. An underground line 39d can be used to feed these extensions so as to provide fluid such as water to these extensions and thus to the containers as well. This irrigation system can also be in communication with computer 420. Computer can also be in communication with another series of computers or controllers as shown in FIG. 14. Computer 420 can be used to read the information received by rain sensor 39b as well as from photovoltaic cells 31 to determine how much water is needed to feed the plants that are provided in containers 22a or 220 along the above listed extensions.

FIG. 3A is a first view of an extension shown as a fence system having an end riser 200a which is a post which provides vertical support for extensions 201a and 202a which are coupled thereto. Inside vertical riser 200a is a fluid conduit 200b. Vertical fence posts 204 are also coupled along this extension to provide vertical support. In this embodiment extensions 201a and 202a provide structural support for containers 22a and 24a or for containers 220 shown in FIG. 3B. Fluid is instead provided through fluid conduits 203a and 204a which provide fluid to these containers. Fluid conduit 200b is in fluid communication with fluid conduits 203a and 204a as well. Fluid conduit 200b is also in communication with underground line 39d. Thus, fluid can be pumped from the water irrigation or sprinkler system of a house or building out to an extension so that the extension can receive a positive flow of water on demand. The pumping controller can be set with a timer to periodically provide water to fluid conduit 200b which then supplies these extensions down the line. Therefore, only one line or zone for a sprinkler system can be set to water all of the extensions down the line.

FIG. 3B shows a side view of this system which shows containers 220 disposed on either side of vertical riser 200a. These containers can be in fluid communication with fluid conduits 203a, 203b, 204a, and 204b. These fluid conduits 200b, 203a, 203b, 204a, 204b can be in the form of a rubber hose, a polymer tubing, piping such as PVC piping or metal piping such as aluminum piping, steel piping or copper piping. The fluid conduits can be any suitable color to either blend in with the fence or with the plants being grown along the extensions. The supply of fluid into these containers is controlled either by a water sensor or a float valve which controls whether a valve is open to receive water into the container or closed to prevent water from flowing into the container.

FIG. 4 shows another embodiment of the invention which discloses another embodiment of an extension which is shown extending having at least two levels for connection to containers such as containers 22a or even containers 220.

In this embodiment 20a there are separate supports which can be any suitable support such as rods or metal piping, or a fence as well. In addition, the support can include vertical supports as well such as vertical supports 25b which extend substantially vertically and connect at connection points 25a and 26c to hold extensions 21A and 23A up above a substantially horizontal plane.

In this view, there is shown containers 22a, 22b, 22c, 22d, which are positioned along line 21a. Line 21a serves as both a holding line and a fluid conduit which holds these containers above a surface as well as provides fluid to these containers. In addition there is shown another set of containers 24a, 24b, and 24c which extend along another line of extensions 21b. Line 21b also serves as a means to support these containers as well as provide fluid to these containers 24a, 24b, and 24c. In this embodiment, line 21a can be in the form of a steel tubing with sufficient thickness to support containers as well as provide fluid to these containers. Disposed along these lines 21a or extensions are quick connect valves which allow containers such as containers 22a, 22b, 22c, and 22d to be fluidly coupled to these lines.

In addition, there are also separate containers 26a which can be positioned on a different level as well. Fluid can be fed from a pumping system 40 which feeds fluid to lines 21a and 21b. The fluid flows into the containers and is controlled by a level sensor which is used to control fluid access to these containers.

FIG. 5 shows a container 22a which includes a fill level sensor 29 which is configured to control access of fluids into each container. In at least one embodiment, the fill level sensor is configured as a float sensor which when the device is filled with water, the fill level sensor rises to a point in the container to close the one way valve, thereby stopping the flow of water into the container. In another embodiment such as shown in FIG. 2C, the fill level sensor is configured as an electronic fill level sensors which is fed by a low voltage source which powers a sensor configured to close a valve in the container to stop fluid flowing into the container.

Thus the flow of water or other type of fluid in the system can be controlled by these successive fill level sensors 29 as shown in FIG. 2B. These fill level sensors can be configured to successively close as each container is set to fill. With a standard irrigation system, a simple connection to a zone of an irrigation system can be used to fill these successive containers in the system. For example, with a first container in the zone, that of container 22a, this container can be filled first with the irrigation system. Once this container is filled, it is then closed by this fill level sensor. Then the fluid is passed onto the next container as well. Each container is then simultaneously or successively filled by this irrigation system so that each container would house the sufficient amount of water to water the plants. For example, each container such as container 22a, can include a fill level sensor including a container 29a, and a float 29b. As container 22a fills with fluid, the separate container which can be formed as a fine mesh container or simply a separate container configured to receive water is configured to fill with water so that the float 29b can float up to a higher level to close a valve 29c. The closing of this valve, thereby closes off water to the system. Once valve 29c is closed, it causes water to flow to the next container and fill that container along the line.

In addition, as shown in this figure, container 22a includes a connector element 27 which is configured to connect to line or extension 21a to support container 22a above a surface.

In this way, each container 22a is filled with a limited amount of water in a way that economizes the amount of water fed to each plant or container. Each container can be oriented so that the plant material can either grow up and out of the container or down and out of the container as well. Alternatively the container can have both ends 28a and 28b open so that the plant material can grow in either direction. In addition coupled to at least one extension such as extension 21a is an optional solar panel 31. Solar panel 31 is configured to receive solar power and to provide power to the system through an electrical line 32 or line 31a extending along the extension 21a. In addition, optionally coupled to solar panel 31 is a communication transceiver 33 which can be configured as either a wireless transceiver or a wired transceiver which is configured to communicate the amount of energy received by solar panel 31 to determine the amount of sun that hits that region of containers 22a. This wireless transceiver can then communicate this information to computer 420 via a wireless router 421 which is in communication with transceiver 33. In addition, there is also an optional rain sensor 37 coupled to transceiver 33 as well as a processing system 35 which is configured to read both the amount of energy received by solar panel 31, as well as read the rain sensor as well. This information is then sent to computer 420 and then onto so as to communicate both the amount of sun and rain hitting that region. Additional solar panels can be positioned around the extensions to provide greater accuracy for server 450 to determine how much sun is hitting each container. All of these components are in communication with each other via line 38 as well. This information for each house or plot can then be cataloged and used to help determine via a connected network how much sun and water is hitting a particular area. As each plot can be cataloged by GPS this system is then configured to provide a highly accurate reading of the amount of rainfall and sun that hits a particular plot.

FIG. 4 also shows a reserve container 30 which can hold a chemical additive or fertilizer agent which can be added to the solution as well. This container can be inserted with this fertilizer or agent to add fertilizer to the solution fed through lines 21a and 21b so that each container can receive fertilizer enriched fluid such as fertilizer enriched water.

FIG. 6A shows a side view of a base connector 50 which is configured to clamp onto an extension or line 201a to secure a container or a container holder thereto. The connector 50 is configured to be coupled via connector elements 52 and 54 which can be in the form of screws having threads which thread through connector and are coupled to extensions such as extensions 201a, and 202a (See FIG. 6D) or extensions 205 in FIG. 6C. As shown in FIG. 6B there is an additional connector 60 which is configured to connect to a container 220 or 22a, 24a etc. This connector includes a strap 62 which can be fed through connecting receivers 64 and 66 which can receive this strap and be configured to clamp the container to this base section 61. This base section 61 can be an arcuate shaped base section for receiving a round container such as container 220, 22a, 24a etc.

FIG. 6D shows a side view of another type of securing configuration wherein a connector element 56 is positioned offset from another connector element 52. This difference in angle a can be such that the connection is substantially transverse, and even in one embodiment substantially perpendicular. This creates connection forces on extension 201a that are substantially transverse to each other to further secure the connector 50 to the extension. This type of connector system can be used with any of the embodiments shown herein.

FIG. 7A shows a top view of another embodiment of container 220. This view shows a container 220 having an outer cylindrical housing 222, spacers 223 and a feeding tube 225 disposed therein. Feeding tube 225 is for receiving a fluid conduit 206 shown in FIG. 8. This spacing by spacers 223 creates a spaced region 226 for receiving fluid such as water therein. An inner housing 224 which is cup shaped or cylindrical is spaced apart from outer housing 222 via spacers. Spacers 223 can be in the form of a cylindrical jacket that is inserted in between the two housings to space these two housings apart or can be formed integral with either outer housing 222 or inner housing 224. Inside of inner housing 224 is an interior region 227 for receiving a plant and plant supporting material such as dirt.

FIG. 7B is a top view of another embodiment. In this embodiment there is shown an addition column 225a which can be substantially cylindrical as well and configured to receive a nutrient supply such as a nutrient stick which can be inserted into this column 225a.

In addition, there is an intermediate housing 228 which is spaced from outer housing 222 via spacers 223 and is spaced from inner housing 224 via spacers 229. Intermediate housing 228 and inner housing 224 can be made porous so that they are configured to receive fluid from outer housing 222 and particularly from the reservoir 226 formed by the spacing of either the inner housing or the intermediate housing from the outer housing. The porous nature of these housings allows for constant fluid communication from the outer regions to the inner regions allowing fluid such as water to flow towards the plant to feed the plant. These housings can be made from a colander type structure, or made from a mesh screen, or made from any other type of porous type structure. These housings 222, 224, and 228 can be made from any suitable material such as plastic, PVC, any suitable polymer, metal, wood, ceramic, composite etc.

FIG. 8 is a side view of the connection system of the container as well as the fluid flow system. For example there is shown a connector which includes a strap 250 for wrapping around an extension 201a. There is also shown receivers 261 and 263 for receiving strap 250 and securing cranks 262 and 264 respectively to crank or turn strap 250 tightly onto extension 201a. This then connects block 235 to extension 201a. Coupled to block 235 is fluid conduit 203a which feeds into valve 205. Valve 205 includes a first section 205a for receiving fluid conduit 203a and an extension element 205b which extends out substantially perpendicularly from first section 205a. Extending down from second section or extension element 205b is a feeding tube 206 which feeds fluid into container such as container 220. Container 220 is shown having a bottom 229a. Straps 233 and 234 are configured to fix the container 220 to block 235 in the same manner as described with strap 250.

FIG. 9A shows a side view of valve 205a which includes turning valve 206a and extension 206b which includes turning section 206b forming a three way valve shown in FIG. 9B.

FIG. 9C shows a quick connect version which shows quick connects 210 for connecting to extension 203a. This view shows valve lever 215 which can selectively open channel 212 as well as channel 212 which is a threaded channel and which can screw into quick connect body section 210 and puncture line 203a to allow water to flow down channel if the valve is open by valve lever 215.

FIG. 10A is a side view of another embodiment. In this embodiment, there is a fence structure 260 which is configured to hold a bracket 270 which has a first wall 275, a top wall 276 and another wall 277 which extends around fence structure 260. Fence structure 260 can be held in place by vertical columns (not shown. On top of wall 276 is a bracket 209 for holding conduit or channel 203 such as 2032. Wall 277 extends down from wall 276 and has lateral extensions 279 extending out laterally from each side (See FIG. 10C) In addition, a plurality of supports comprising loops 272 and 274 are coupled to this bracket 270 and are configured to hold a container in place. This container can slide therein to these supports and rest inside these supports.

FIG. 10B is a side view of another embodiment of a support 280 which shows a cross-sectional view of a fence support structure 260. In this view there is a wall 285, another wall 286 and a front wall 287, however coupled to this front wall 287 is a spacer 288 which is configured to support this front wall 287 away from a back of a fence. Thus, depending on which side of the fence that this device is positioned on, this spacer, 288 which is coupled to side wings 289 is used to support front face 287 against the rotational movement of the bracket 285, 286 and 287 once it is set upon fence support structure 260. In addition there is shown supports 282 and 284 which are configured to support a container therein. Furthermore, there can be a bracket 209 for holding a conduit or channel 203 such as channel 203a which is used to propel water to these containers.

FIG. 10C is a front view of the support 280. In this view, there is shown conduit 203, supported by bracket 209. In addition there is shown support loops 282, and 284 which are configured to support a container therein. In addition, there is shown front wall 287 which extends down from the fence support structure. In addition extending laterally out from this front face 287 are wings or lateral or rotational stabilizes 279, 289 which extend laterally out from front face 287. These lateral stabilizers 279 and 289 are used to support the structure against rotation when a container is inserted therein.

FIG. 11 is a side view of an indoor tray which can be used to grow plants in an initial type system. For example, this tray system 350 can be used to grow smaller types of plants such as herbs, or initial shoots of plants which can then be individually inserted into individual containers 22a, 22b, 22c, 24a, 24b, 24c etc.

This tray can include a manifold 352 which includes a plurality of successive holes 354a, 354b, 354c, 354d, 354e, and 354f as well as a plurality of corresponding channels 355a, 355c, 355d, 355e, 355f. In addition, along each channel such as channel 355a there are a plurality of openings 356a, 356b, 356c etc. which are contained in a sheet 356.

Manifold 352 is fed by a pump (not shown) and fluid then follows through opening 354a for example, and down channel 355a to successively feed fluid to any plant material which is housed in any one of openings 356a.

FIG. 12A shows a side view of an opening or hole 354a which is configured to receive a cartridge 359 or similar type of element which is configured to hold a plant seedling or herb sprout. At the hole 354a, there is a gasket 357 which is configured to receive these types of cartridges. This cartridge is configured to receive and/or hold, at least one seed or seedling, nutrients such as fertilizer, as well as a medium for growing plants such as rocks or dirt.

FIG. 13 is a cross-sectional view of tray system 350 which shows a container 360 having a body 363, which has containers 364, and 380 having pipes 367 and 382 to feed into a water feed tube 385 and which allows water to flow down channels such as channels 355a into a catch basin pouring through line 368 into another catch basin wherein the water goes through line 367 and back into basin 364. Pump 370 pumps water through the tube 385 to the top of the tray 350. The tray 350 is configured to sit at an angle to allow water to flow down the front of the tray and then cycle back through the system through line 368.

FIG. 14 is a plan view of a computer system which is used to control and plan for the system shown in FIGS. 1-13. For example, with this system, there are a plurality of personal computers, remote computers, iphones, or any other types of portable or stationary computing devices 410, 420, and 430 etc. As shown in FIG. 15 each of these devices includes a processor such as a microprocessor, 420a, a memory such as a ram 420b, a mass storage device 420c, a transceiver 420d, a power supply 420e, and a motherboard 420f. Each of the other computing devices also include these components as well. For example, FIG. 15B includes these components as well including microprocessor 450a, memory 450b, mass storage device 450c, transceiver 450d, and power supply 450e, as well as motherboard 450f.

For example, the three different servers 440, 450, and 460 are configured to carry out the process for selecting and organizing the planting system and to carry out the steps indicated in FIGS. 5-6. In at least one embodiment server 450 is an application server which is configured to perform the process disclosed in FIGS. 16 and 17.

For example, FIG. 16 shows the process for selecting the types of plants to plant in any one of containers 220 or containers 22a, 22b, or 26a etc, or to plant in any one of cartridges 359 into holes 354a etc. This process can be performed using any type of server on a computer network such as via server 450 using microprocessor 450a and communicating to other computers via the internet 400. This communication can be via a transceiver 450d such as using a network interface card and communicating via Ethernet lines. Other types of communications or communication protocols can also be used.

For example, in step S1 a user can log into the system such as an applications server 92 by providing his or her login information such as name, and password. This is performed via a webpage which allows information to be uploaded. Next, the logged in user can input his or her geographic information. This can be performed by inputting the user's address including zip code or any other type of geographic information. If the user is using a mobile computing device, that device can include a GPS device which can be configured to set the location of the plantings or the lot of the user such as the lot as shown in FIG. 1. Thus, in step S2 once this geographic information is logged, a user can set the plot lot, and the dimensions of the plot lot such as shown in FIG. 1. For example, the user can set the dimensions of the lot and the orientation of the lot. For example, the user can set the way that the lot faces with respect to the house and shade on the lot. Therefore, if a house faces south, then the front end of the house would have the best sun, the right side of the house would be the east side, the back side of the house would be the north side, and the left side of the house would be the west side. Accordingly the plants can be planted based upon the level of sun that would be present based upon the shade provided by the house or any other structure or planting on the lot.

Thus the orientation and the plotting of the lot can be configured so as to indicate the amount of sunlight that is provided to each container on the lot.

Next, in step S4 the user plots the containers on the lot as well as the lines which are configured to feed these containers. The position of each container would then be logged to indicate the amount of sun and water that would be available to each container. The user would then in step S5 input the water availability to each of these containers as well. This water availability is configured based upon the lines that are present along the plot and the volume of fluid that can be delivered to each container.

Next, in step S6 the system would log additional information corresponding to weather and water from neighbors who are logged into the system. Next, in step S7 the system would present on a web page a series of questions to the user to ask which types of plants that the user would like to grow. For example, the system could present questions relating to the types of fruits and vegetables that the user likes to eat.

Next, in step S8 the system would log this information into a database such as through a database server 94 and then in step S9 apply an algorithm which would suggest which types of plants to use. For example, this algorithm would first select a plant based upon the geographic region that the plot was located, and then select the plant based upon the time of year for planting, and then base the selection based upon the position of the container for the plant in the lot, and then select the plant based upon the amount of expected sunlight based upon any shade present on the lot. Additional factors that can be included can also be the information provided by neighbors and their successes in growing these plants as well. Next, the system would apply that list of feasible plants and compare the feasible plants to the plants suggested by the user to find any matches.

The order or hierarchy in which the above criteria are applied can be in any suitable order. Once matches are provided, the system can provide a list of plants that the user can use to plant in each of the containers. Included in this list of plants can be a ranking of suggested plants that the user should consider. This ranking can be based upon the feasibility of planting these plants based upon the weather conditions, the time of year of planting, the amount of suitable sunlight etc.

Once the user has his or her list, the system can plot out how to plant these plants in the garden. For example, as shown in FIG. 17, in step S12 the system can categorize each of the containers for planting by either grouping them in groups or at least indicating which containers are suitable with certain types of plants

Thus, in step S13, the system would match the position of each container with each container.

Next, in step S14 the system would match the plants selected by the user with the most suitable containers selected based upon the position of each container on the lot, as well as the size of the container indicated as well. For example, if a container is positioned in a southerly exposure which receives a lot of sunlight and the planting was to occur in June, the system may suggest planting tomatoes if tomatoes are indicated as being particularly suitable plantings for such as container.

Next, in step S15, the system would set the time for each of the plantings. For example, if tomatoes were set to grow and become ripe in 6 weeks on average if they are planted in the 11030 area code in on June 6, then if tomatoes were planted in the first container 22a which has a substantially southerly exposure then the system would start a timer to indicate that the tomatoes would likely become ripe within six weeks.

Next, in step S16 the system would set the suggested water delivery such as 3× a day or a certain volume of water that should be provided to each container. In this case, the system could even suggest the position for the placement of the fill level sensor 29 in each container based upon the plant being planted in each container.

Next, in step S18 the system could monitor the sun delivery to the system to provide an account over time of the sun delivered to each container in the system. This monitoring of sun delivery can be in the form of reading a solar panel and determining the amount of energy produced by the solar panel across a single time period such as a day to determine the amount of sun delivery to a region.

Next in step S19 the system could monitor the water delivery by logging the weather or recording the amount of water that has fallen on the plot. Next, in step S21, the system could apply an algorithm to determine based upon the weather, including the amount of rain as well as the amount of sun or the heat over a period of time to readjust the time for harvest.

Next, in step S22 the system could indicate when to harvest the plants. This indication could be in the form of an email, or other type of suitable notification. Next in step S23 the user could harvest the plants wherein the user could either remove the plants or place the plants in another container for planting.

Accordingly, while at least one embodiment of the present invention have been shown and described, it is to be understood that many changes and modifications may be made thereunto without departing from the spirit and scope of the invention as defined in the appended claims.

What is claimed is:

1. An irrigation device comprising:
a) at least one holding bracket;
b) at least one conduit bracket, configured to hold a fluid conduit;
c) at least one front face extending down from said bracket;
d) at least one support for supporting at least one container, said support being coupled to said at least one front face;
e) at least one lateral extension extending out from said at least one front face, said at least one lateral extension having at least a first lateral extension on a first side and at least a second lateral extension on a second side.

2. The irrigation device as in claim 1, further comprising at least one container configured to receive water therein, said at least one container having a float valve, wherein said float valve is configured to close when water in the container reaches a predetermined level.

3. The irrigation device as in claim 1, further comprising a fluid conduit and coupled to said at least one conduit bracket, wherein said fluid conduit is in communication with said at least one float valve.

4. The irrigation device as in claim 3, further comprising at least one additional container coupled to said fluid conduit and coupled in series with said fluid conduit so that when water in said first container fills to a predetermined level, the remaining water flows into at least said at least one additional container,

5. The irrigation device as in claim 2, further comprising at least one spacer configured to fit inside of said at least one container.

6. The device as in claim 5, wherein said at least one container is substantially cylindrical.

7. The device as in claim 6, wherein said at least one spacer comprises at least one cylindrical spacer configured to create a narrow opening in said container.

8. The device as in claim 1, further comprising a fluid conduit extending in said container between said at least one spacer and a wall of said conduit.

9. The device as in claim 8, further comprising at least one float valve disposed in said fluid conduit.

10. A portable vegetation growing system comprising:
a) a base;
b) at least one irrigation channel coupled to said base;
c) at least one platform having a plurality of openings disposed therein and configured to receive at least one cartridge of material, said cartridge comprising at least one seed, at least one fertilizer and at least one medium for plant growth.
